(19) United States
(12) Patent Application Publication
(10) Pub. No.: US 2012/0115191 A1
(43) Pub. Date: May 10, 2012

(54) PRODUCTION OF FRUCTO-OLIGOSACCHARIDE AND DERIVATIVES BY USE OF ASPERGILLUS SPP

(75) Inventors: Samir S. Somaiya, Mumbai (IN); Sangeeta Srivastava, Mumbai (IN); Martin Annette, Mumbai (IN); Amol Dive, Mumbai (IN)

(73) Assignee: Godavari Biorefineries Ltd.

(21) Appl. No.: 13/203,566

(22) PCT Filed: Feb. 28, 2010
(86) PCT No.: PCT/IN2010/000118

§ 371 Date: Aug. 26, 2011

(30) Foreign Application Priority Data
Feb. 27, 2009 (IN) 448/MUM/2009

(51) Int. Cl. C12P 19/00; C12P 19/24; C12N 1/14

(52) U.S. Cl. 435/94; 435/256.1; 435/72

(57) ABSTRACT

There is provided a new strain, that is Aspergillus fumigatus, MTCC 5453, and its use in the production of fructo-oligosaccharide. The invention also provides an efficient, rapid and method for increased production of fructo-oligosaccharide by using the strain Aspergillus fumigatus MTCC 5453.

### FIELD OF INVENTION

The present invention relates to the method for producing Fructo-oligosaccharides (FOS) and/or derivative thereof. In particular the invention further relates to production of fructo-oligosaccharides and/or derivative thereof by use of a new non pathogenic micro-organism, Aspergillus fumigatus. More in particular the invention relates to the efficient, rapid and increased production of fructo-oligosaccharide and/or derivative thereof.

### BACKGROUND OF THE INVENTION

Fructo-oligosaccharides also called oligofructose, oligofructan, Neosugar or Inulin is a complex sugar of the class of oligosaccharides used as artificial and/or alternative sweetener. The term oligosaccharide refers to a short chain of sugar molecules and accordingly Fructo-oligosaccharides comprises of short chain of fructose molecules.

Fructo-oligosaccharides are derived from plants and are very good for the human body in contrast to the commonly used refined sugar. Fructo-oligosaccharides are transported directly to the gut, where bacteria aid in their digestion. They thus act like fiber, passing undigested to the large intestine where they are fermented by colonic bacteria. Subsequently there is a growth spurt among the populations of bifidus bacteria (e.g. Bifidobacteria and lactic acid bacteria) that eventually outgrow the pathogenic and/or the putrefactive bacteria (e.g. colon bacilli). Therefore there is a decrease in toxic fermentation products. Fructo-oligosaccharides also aid in lowering the cholesterol and blood sugar levels, and enhance absorption of the mineral elements.

Fructo-oligosaccharides can be found in large quantity of foods found in nature, such as asparagus, banana, garlic, onion, tomato or wheat. Vegetables like artichoke, burdock, chicory, leeks, onions, and asparagus and fruits like Jerusalem artichokes are considered very good sources of fructo-oligosaccharides. These sugars find their application in more than 500 kinds of food and pharmaceutical products in many countries and are widely used in USA, EU and Japan.

In view of the benefits described above, the interest for production of fructo-oligosaccharides have raised progressively. Efforts are evident from the various studies that have focused on the production of the fructo-oligosaccharides. The U.S. Pat. No. 7,063,976 describes the process of obtaining beta-fructofuranosidase enzyme and a process for producing fructo-oligosaccharides. The said process involves first preparing the beta-fructofuranosidase enzyme and then obtaining the fructo-oligosaccharides using the said enzyme, wherein the preparation of enzyme itself takes about 4-10 days. Thus, the said process is complex and very lengthy, hence may not be suitable at an industrial scale.

Similarly U.S. patent application Ser. No. 20050069627 discloses the process for preparation of fructo-oligosaccharide by the reaction of extra cellular Fructosyl Transferase (FTase) enzyme obtained from Aspergillus species on the substrate (sucrose, jaggery optionally along with stevia extract) under fermentation conditions. Sangeetha et al showed fructo-oligosaccharide production upto 58% using fructosyl transferase (FTase) produced by Aspergillus oryzae CFR 202 under submerged fermentation conditions. These processes are complex and lengthy too, as they involve the preparation of enzyme first, which is then utilized for the production of fructo-oligosaccharides, wherein the preparation of enzyme takes minimum 4 days. Besides, the fermentation needs to be carried out at higher temperature, making process energy intensive and more costly.

Further, Sanchez et al showed that sucrose can be rapidly converted into fructo-oligosaccharide and glucose, increasing the initial sucrose concentration increased the fructo-oligosaccharide production, the said method involves use of highly specialized reactor with specific membrane. The process is carried out at very high temperature. Thus, the method would be expensive and industrially not suitable. Likewise Hidaka et al reported synthesis of fructo-oligosaccharide in concentrated sucrose solutions which was attributed to the transfructosylating action of enzyme beta-f ructofuranosidases on sucrose. Thus, this method also suggests first preparation of the said enzyme and then conversion of sucrose into fructo-oligosaccharide. Further, the fructo-oligosaccharides is produced at the higher temperature which makes the process energy intensive. The process also requires longer fermentation duration. Katapodis et al observed biosynthesis of fructo-oligosaccharides during growth of the thermophilic fungus Sporotrichum thermophile on media containing high sucrose concentrations. The said methods suffer from the disadvantages such as very low concentration of fructo-oligosaccharides produced, utilization of high cost nutrients such as amino acids, rendering the process industrially not feasible. Dorta C. et al. discloses the process for fructo-oligosaccharide production by Aspergillus japonicus (FCL 119T) and Aspergillus niger (ATCC 20611) using Sugarcane molasses and yeast powder. The said process needs two separate strains, despite it produces very low concentration of fructo-oligosaccharides. Thus, the strains and methods described above have various shortcomings including low conversion rate of fructo-oligosaccharide and too complex and lengthy processes. Therefore, they are not suitable for massive industrial production.

Based on the current status of fructo-oligosaccharide production, fructo-oligosaccharide supply and the FOS demand, there is a need for alternative and more efficient methods for higher and more rapid production of fructo-oligosaccharides.

### DISCLOSURE OF THE INVENTION

It is an object of the present invention to provide a method of producing Fructo-oligosaccharides and/or derivative thereof by the use of the new non pathogenic micro-organism Aspergillus fumigatus MTCC 5453.

It is further object of the present invention to provide an efficient, rapid and increased production of FOS and/or derivative thereof.

### SUMMARY OF THE INVENTION

The present invention relates to the method for producing fructo-oligosaccharides and/or derivative thereof by use of a non pathogenic micro-organism, Aspergillus fumigatus MTCC 5453. In particular, the invention relates to the micro-organism, the Aspergillus fumigatus MTCC 5453 and a cell culture comprising the micro-organism. The invention also relates to an efficient, rapid and increased production of FOS and/or derivative thereof.

### DETAILED DESCRIPTION OF THE INVENTION

The present invention provides a method for production of fructo-oligosaccharide and/or derivative thereof, by the microorganism Aspergillus fumigatus MTCC 5453 comprising culturing the micro-organism according to the invention.

The fructo-oligosaccharide “derivative” as used herein refers to any compound modified in atleast one chemical element and/or group compared to fructo-oligosaccharide of the invention. However, the derivative expresses a biological activity similar to and/or the same as that of fructo-oligosaccharide. The fructo-oligosaccharide derivative may be obtained by further modifying the strain according to the invention and/or modifying atleast one step of the process of fructo-oligosaccharide preparation. Accordingly, any reference to fructo-oligosaccharide encompasses any possible fructo-oligosaccharide derivative and/or similar compound.

According to one aspect of the present invention there is provided a micro-organism, the Aspergillus fumigatus MTCC 5453.

A further aspect of the current invention provides a cell culture comprising the micro-organism Aspergillus fumigatus MTCC 5453.

In one embodiment, the present invention provides a cell culture comprising the microorganism Aspergillus fumigatus MTCC 5453 suspended in any conventional or known culture medium suitable for Aspergillus spp., preferably the medium is fermentation medium as described hereinafter.

According to yet another aspect the present invention provides a method for the production of fructo-oligosaccharide and/or a derivative thereof comprising culturing of the micro-organism Aspergillus fumigatus MTCC 5453 in fermentation media.

The method according to the invention comprises incubating the micro-organism in a fermentation medium so as to produce and concentrate fructo-oligosaccharide the cells of the micro-organism and/or the medium. The method may further comprise collecting and/or purifying the fructo-oligosaccharides from the cells and/or medium.

The method according to the invention has shorter cycle, lower cost, and higher production rate. Further, it allows an efficient method for mass production of fructo-oligosaccharide and thus may be used in industry. Its industrial application can not only protect natural resources, but satisfy the requirement of the clinical drug demand as well.

In one embodiment, the present invention provides a method for the production of fructo-oligosaccharide and/or a derivative thereof comprising culturing of the micro-organism Aspergillus fumigatus MTCC 5453 in the fermentation media may concentrate the fructo-oligosaccharide in the cells of the micro-organism and/or the fermentation media. The method may further comprise collecting and/or purifying the fructo-oligosaccharide from the cells of micro-organism and/or the fermentation media. The method of the production of fructo-oligosaccharide and/or a derivative thereof may be by fermentation of carbon source. The fermentation may be carried out at 25-30° C. and/or at a pH of 5-6 for atleast 15 hours, preferably 15-30 hours.

Aspergillus fumigatus was isolated from the soil samples collected from the vicinity of Sakarwadi distillery, located at, Ahmednagar, Maharashtra, India. The fungal spores were grown in a culture medium under stirring conditions to obtain an inoculum. The culture medium may be any conventional or known culture medium suitable for Aspergillus spp., preferably the medium is fermentation medium as described hereinafter. The strain was identified as Aspergillus fumigatus. This species has been deposited with Microbial Type Culture Collection and Gene Bank (MTCC), at Institute of Microbial Technology (IMTECH), and its address is Institute of Microbial Technology, Sector39-A, Chandigarh, India. The deposit number is MTCC 5453.

The new strain Aspergillus fumigatus MTCC 5453 provided by the present invention was identified and distinguished from the conventional strain and details are as given in

TABLE I
Table 1
Observed Biochemical Chart

| | Colony characteristics | | Microscopic | | |
|---|---|---|---|---|---|
| | on Sabourauds agar | | Conidiophore | | Identified |
| No | Surface | Reverse | Features | Hyphae | strain |
| 1 | Blue green | White to | Short smooth | Septate | Aspergillus |
| | to Grey | tan | greenish | | fumigatus |

The new strain Aspergillus fumigatus MTCC 5453 of the present invention is a non pathogenic strain. Aspergillus fumigatus MTCC 5453 showed no zone of inhibition when treated with berberine sulphate, as per the method reported in J. Med. Microbiol.—Vol. 50 (2001), 653-654, confirming that the Aspergillus fumigatus MTCC 5453 is non-pathogenic strain.

The invention provides a new method to produce fructo-oligosaccharide with the new strain Aspergillus fumigatus MTCC 5453 of the present invention. The method comprises culturing the present invention strain Aspergillus fumigatus MTCC 5453 in a fermentation medium to produce and concentrate fructo-oligosaccharide in the cells of said strain and/or the medium, and collecting and purifying fructo-oligosaccharide from the said cells and/or medium.

The “fermentation medium” may be any conventional culture or fermentation media used for Aspergillus spp. or any known media in the field. The fermentation medium may comprise carbon source and nitrogen source and/or carbon source. Other organic and/or inorganic materials may be added into the fermentation medium so as to accelerate growth of the microorganism and increase the rate of fructo-oligosaccharide production.

In one of the embodiment, the present invention provides a fermentation media comprising:
carbon and nitrogen source and/or carbon source
micro-nutrients and/or trace minerals at favourable pH and/or temperature.

The fermentation media may further comprise glucose, sucrose, maltose, fructose, glycerol, starch, lactose and/or galactose for growth of Aspergillus fumigatus. The carbon source may be selected from the group consisting of sugarcane juice, jaggery, molasses, sugarbeet, wheat, jowar and/or sweet sorghum, wherein the carbon source may be atleast 10% (w/v), atleast 20% (w/v), atleast 30% (w/v), atleast 40% (w/v), atleast 50% (w/v). The carbon source may be 60% (w/v). The Nitrogen source may be selected from the group consisting of peptone, yeast extract, casein, malt extract, powder of peanut, powder of soybean, corn steep solid, yeast powder, ammonium nitrate, and/or ammonium chloride and/or Soya, wherein the nitrogen source may be atleast 0.1% (w/v), atleast 0.25% (w/v), atleast 0.5% (w/v), atleast 1% (w/v).

The fermentation medium may further comprise inorganic nutrients like phosphate, magnesium salts such as magnesium sulphate, ferric salts such as ferrous sulphate, ferric chloride, potassium salts such as potassium di hydrogen phosphate but not limited to these. The fermentation medium may further comprise, but limited to, trace minerals, such as boric acid, potassium iodide, zinc sulphate, manganese sulphate. The medium may also comprise, inducers, such as methyl jasmonic acid, arachidonic acid, aminocitrate, ceric ammonium nitrate, potassium permanganate, pyruvic acid, p-coumaric acid, vanadic sulfate, hippuric acid, α-naphthylacetic acid, 6-benzylamine purine, silver nitrate, and cinnamic acid but should not be considered to be limiting to these. The fermentation medium may also comprise but not limited to precursors, such as-phenylalanine, benzamide, sodium benzoate, sodium acetate, propionamide, benzoic acid, and acetamide.

The culture may be under conventional and/or known fermentation conditions in the art. The fermentation may be under aerobic conditions at the temperature of atleast 25° C. In particular the temperature may be in the range of atleast 25-30° C. The fermenting pH value may be atleast 5.0. In particular the pH may be in the range of 5.0-6.0. The pH value may be adjusted in the mid and late phases of fermentation.

The fermentation may be carried out using known apparatus, conditions and/or methods known in the art, for example, oscillating the bottle under a speed rate known for the particular apparatus, condition and/or method. The fermentation may also be carried out in conventional fermentation tanks, such as the 7L and 50L tanks. A 10% spore suspension of Aspergillus fumigatus MTCC 5453 may be prepared in fermentation media and the same media may be used for passages.

In order to concentrate fructo-oligosaccharide from the fermentation medium, any methods known in the art may be used. For example, after fermentation is completed, the fermentation medium is filtered, the mycelia is discarded. The filtrate comprising the fructo-oligosaccharide may be further distilled and purified using methods known in the art, such as charcoal treatment, chromatography and/or crystallization, but may not be limited to these methods.

The method of collecting and/or purifying fructo-oligosaccharide may comprise
separating the mycelia from the fermented broth;
Distilling the filtrate and obtaining fructo-oligosaccharide.

The fructo-oligosaccharide produced may be concentrated in the form of gel by methods known in the art. The concentration may be analyzed by standard techniques such as HPLC. Fructo-oligosaccharide produced by the new strain Aspergillus fumigatus MTCC 5453 of the invention mainly exists in the fermentation medium but may also be present intracellularly. The fructo-oligosaccharide produced is with increased fermentation rate per liter. The concentration of fructo-oligosaccharide gel obtained by the method of the present invention may be 45-60%. The present invention gives a yield of atleast 70%.

The method may further comprise the production of fructo-oligosaccharide and/or a derivative thereof by conversion of carbon source by fungal enzymes produced by the micro-organism Aspergillus fumigatus MTCC 5453. In particular the enzymes may be selected from the group consisting of, but not limited to fructan hydrolysing enzyme comprising invertase and/or levanase, and/or isomerase.

The fructo-oligosaccharide produced by the method of the invention may further comprise other favourable characteristics in humans such as ability to
1. prevent constipation and diarrhea
2. lower blood fat
3. Increase immunity
4. Prevent obesity
5. Increase the synthesis of vitamin B and improve absorption of mineral elements.

Further this oligosaccharide may be used as a sugar replacement, dietary supplement and/or food additives.

The use of the new strain Aspergillus fumigatus MTCC 5453 in the production of fructo-oligosaccharide is capable of producing fructo-oligosaccharide, wherein the yield of fructo-oligosaccharide is atleast 70%. An essential feature of the invention is that, the strain Aspergillus fumigatus MTCC 5453 in culture is capable of higher fermentation rate, thus capable of producing higher concentration of fructo-oligosaccharide. Therefore the current invention solves an essential problem in the field and offers an advantage over the microbial fermentation methods available in the prior art. The technology of the current invention may be characterized by a short cycle, low cost, and high fermentation rate. The method gives increased yield of atleast 70% fructo-oligosaccharide in 24 hrs. Therefore the fructo-oligosaccharide production method described herein is capable of being applied for industrial production. The use of non pathogenic strain contributes to protecting the natural environments yet being able to meet the demand.

While the invention has been described with particular reference to certain embodiments thereof, it will be understood that various modifications can be made to the above-mentioned embodiments without departing from the spirit and scope of the present invention. The examples and the particular proportions set forth are intended to be illustrative only.

### EXAMPLES

### Example 1

The fungal strain Aspergillus fumigatus (MTCC 5453) spores were cultured in 5 ml fermentation medium comprising 30% sucrose and 0.5-1.0% peptone along with micro nutrients including 0.5% KH₂PO₄, 0.01% ZnSO₄, 0.5% MgSO₄ with 0.001% polysorbate 80 solution at pH 5.3 and temperature 26° C. under stirring conditions to obtain an inoculum of starter culture. 5 ml inoculum was used to inoculate a larger volume of 45 ml into the 250 ml flask containing 45 ml fermentation medium for 25hrs. The 50 ml inoculum was further inoculated in 1000 ml flask containing 450 ml fermentation media for 24 hrs at pH 5.3. After fermentation, the broth was filtered and was concentrated by conventional method to form a gel. The gel was analyzed by HPLC. The concentration of fructo-oligosaccharides was 45% with yield of 75%.

### Example 2

The fungal strain Aspergillus fumigatus (MTCC 5453) spores were cultured in 10 ml fermentation medium comprising 40% sucrose and 0.5-1.0% peptone along with micro nutrients including 0.25% KH₂PO₄, 0.1% FeSO₄, 0.5% MgCl₂ with 0.001% polysorbate 80 solution at pH 5.6 and temperature 27° C. under stirring condition to obtain an inoculum of starter culture. The 10 ml inoculum was used to inoculate a larger volume of 90 ml into the 250 ml flask and allowed to ferment for 27 hrs. The 100 ml inoculum was further scaled up in 5000 ml flask containing 900 ml fermentation media for 27 hrs at pH 5.6. After fermentation, the broth was filtered and was concentrated by conventional method to form a gel. Gel was analyzed by HPLC. The concentration of fructo-oligosaccharide was 55% with yield of 78%.

### Example 3

The fungal strain Aspergillus fumigatus (MTCC 5453) spores were cultured in 15 ml fermentation medium comprising 50% sucrose and 0.5-1.0% casein along with micro nutrients including 0.1% KH₂PO₄, 0.001% ZnSO₄, 0.1% MgSO₄ with 0.0001% polysorbate 80 solution at pH 5.8 and temperature 29° C. under stirring condition to obtain an inoculum. The 10 ml of the above inoculum was inoculated into 250 ml flask containing 90 ml of fermentation media for 29 hrs. The 100 ml inoculum was inoculated in 5000 ml flask containing 900 ml fermentation media for 29 hrs at pH 5.8. For further inoculum development, 250 ml inoculum was added to 5000 ml flask containing 2250 ml of fermentation media in four sets respectively. 10 liter inoculum was added to 100 liter reaction vessel containing 90 liter of fermentation media. The pH was maintained at 5.8. After fermentation, the broth was filtered and was concentrated by conventional method to form a gel. Gel was analyzed by HPLC. The concentration of fructo-oligosaccharide was 60% with yield of 80%.

### REFERENCES

Hidaka, H., M. Hirayama and N. Sumi; 1988, Agric. Biol. Chem. 52:1181-1187 Dorta, C., Cruz, R., Oliva-Neto, P. and Camargo, D. J.; 2006, J. Ind. Microbiol. Biotechnol. 33:1003-1009

Katapodis P., Kalogeris E., Kekos D., Macris B 3., Christakopoulos P.; 2004, Applied Microbiology and Biotechnology, 63, 378-383,

Sangeetha P T, Ramesh M N, Prapulla S G; Appl Microbiol Biotechnol.; 2004; 65(5):530-7. Epub 2004 Jun. 25

Sánchez, O. F., 2006, Study of fructosyltransferase production by an Aspergillus sp. native strain from sucrose in a bench-scale membrane reactor. M.Sc. Chemical Engineering Thesis, Universidad Nacional de Colombia, Sede Bogotá. (in Spanish).

1. A micro-organism, the Aspergillus fumigatus MTCC 5453.

2. A cell culture comprising the micro-organism according to claim 1.

3. A method for the production of fructo-oligosaccharide and/or a derivative thereof comprising culturing of the micro-organism Aspergillus fumigatus MTCC 5453 in fermentation media.

4. The method according to claim 3, wherein the fermentation media comprises
carbon and nitrogen and/or carbon source
micro-nutrients and/or trace minerals at favourable pH and/or temperature.

5. The method according to claim 3, wherein the fermentation media further comprises glucose, sucrose, maltose, fructose, glycerol, starch, lactose, galactose, powder of peanut, powder of soybean, corn steep solid, yeast powder, peptone, yeast extract, ammonium nitrate, and/or ammonium chloride for growth of Aspergillus fumigatus MTCC 5453.

6. The method according to claim 4, wherein the carbon source is selected from the group consisting of glucose, sucrose, maltose, fructose, glycerol, starch, lactose, galactose, sugarcane juice, jaggery, molasses, sugarbeet, wheat, jowar and/or sweet sorghum.

7. The method according to claim 6, wherein the carbon source is in the range of 10-60% (w/v).

8. The method according to claim 4, wherein the Nitrogen source is selected from the group consisting of peanut, powder of soybean, corn steep solid, yeast powder, peptone, yeast extract, ammonium nitrate, and/or ammonium chloride, casein, malt extract and Soya.

9. The method according to claim 3, wherein the culturing of the micro-organism Aspergillus fumigatus MTCC 5453 in the fermentation media concentrates the fructo-oligosaccharide in the cells of the micro-organism and/or the fermentation media.

10. The method according to claim 9, further comprising collecting and/or purifying the fructo-oligosaccharide from the cells of micro-organism and/or the fermentation media.

11. The method according to claim 3, wherein the production of fructo-oligosaccharide and/or a derivative thereof is by fermentation of carbon source.

12. The method according to claim 11, wherein, the fermentation is carried out at 25-30° C. and/or at a pH of 5-6.

13. The method according to claim 11, wherein, the fermentation is carried out for atleast 15-30 hours.

14. The method according to claim 3, wherein, the production of fructo-oligosaccharide and/or a derivative thereof is by conversion of carbon source by fungal enzymes produced by the micro-organism Aspergillus fumigatus MTCC 5453.

15. The method according to claim 14 wherein, the enzymes are selected from the group consisting of fructan hydrolysing enzyme comprising invertase and/or levanase, and isomerase.

16. The method according to claim 10, wherein the steps of collecting and/or purifying fructo-oligosaccharide comprises
separating the mycelia from the fermented broth;
Distilling the filtrate and obtaining fructo-oligosaccharide.

17. The method according to claim 16, wherein the concentration of fructo-oligosaccharide obtained is 45-60%.

18. The method according to claim 3, wherein the yield of fructo-oligosaccharide obtained is atleast 70%.

19. A method for the production of fructo-oligosaccharide and/or a derivative thereof substantially as herein described with reference to forgoing description and examples.
